(19) United States
(12) Patent Application Publication
(10) Pub. No.: US 2004/0023298 A1
(43) Pub. Date: Feb. 5, 2004

(54) Method for measuring jnk/sapk activity by phosphorylation of transcription factors

(76) Inventors: Ingrid Herr, Ziegelhausen (DE); Klaus-Michael Debatin, Ulm (DE)

Correspondence Address: INTELLECTUAL PROPERTY / TECHNOLOGY LAW, PO BOX 14329, RESEARCH TRIANGLE PARK, NC, 27709, US

(21) Appl. No.: 10/343,068

(22) PCT Filed: Jul. 24, 2001
(86) PCT No.: PCT/DE01/02707

(30) Foreign Application Priority Data
Jul. 25, 2000 (DE) 100 36 501.9

(51) Int. Cl. G01N 33/53

(52) U.S. Cl. 435/7.1

(57) ABSTRACT

The invention relates to a method for measuring the JNK/SAPK activity which is based on the implemation of a kinase reaction with JNK/SAPKs which are obtained from a cell lysate and preferably isolated by immune precipitation in the presence of ATP and GST transcription factors (preferably: GST-c-Jun), and subsequently measuring the amount of phosphorylate GST transcription factors (preferably: p-GST-c-Jun) according to the specific binding of an anti-p-GST transcription factor antibody (preferably: anti-p-GST-c-Jun-antibody).

The present invention relates to a method of determining JNK/SAPK activity, which is based on the implementation of a kinase reaction with JNK/SAPKs obtained from a cell lysate in the presence of ATP and GST transcription factors and subsequently determining the amount of phosphorylated GST transcription factors (p-GST transcription factors) by the specific binding of an anti-p-GST transcription factor antibody.

c-Jun-NH₂-terminal kinases (JNS) which are also known as SAPK (stress-activated protein kinases) are signal transduction proteins which can be activated under the most different conditions (Davis, Biochem. Soc. Symp. 64 (1999), 1-12; Gupta et al., Science 267 (1995), 389-393). JNK/SAPKs regulate the gene expression by means of phosphorylation and, as a result, the activation of transcription factors, such as c-Jun (Davis, Biochem. Soc. Symp 64 (1999), 1-12; Minden and Karin, Biochim. Biophys. Acta 1333 (1997), 85-104; Herr et al., EMBO J. 16 (1997), 6200-6208). Thus, the phosphorylation state of the transcription factors, e.g. of c-Jun, reflects the activity of JNK/SAPKs and the common assays for determining this activity are also based thereon. In these JNK/SAPK assays, JNKs are immunoprecipitated from cell lysates, and an in vitro kinase assay is carried out by incubation of the purified JNKs together with GST-c-Jun protein and ³²P-y-ATP. SDS-PAGE and autoradiography serve for determining the amount of phosphorylated GET-c-Jun protein which reflects the activity of the JNK/SAPKs (Hibi et al., Genes Dev. 7 (1993), 2135-2148). However, these assays have the following drawbacks: They are based on radioactive labelings, are rather insensitive, time-consuming and have a high background.

Thus, the present invention is based on the technical problem of providing a JNK/SAPKs assay which avoids the above summarized drawbacks, i.e. does not require radioactive labeling and is additionally sensitive in spite of the little time spent (exposure time).

The solution to this technical problem is achieved by providing the embodiments characterized in the claims.

During the experiments which led to this invention it turned out that the above described problems can be overcome by determining the activity of JNK/SAPKs by carrying out a kinase reaction with JNK/SAPKs obtained from a cell lysate in the presence of ATP and GST transcription factors, and then by determining the amount of phosphorylated GST transcription factors (p-GST transcription factors) by the specific binding of an anti-p-GST transcription factor antibody, the amount of bound antibody reflecting the amount of phosphorylated GST transcription factor (p-GST transcription factor) and thus indirectly the activity of JNK/SAPKs.

Thus, the present invention relates to a method of determining the activity of JNK/SAPKs, the method comprising the steps of:

(a) preparing a cell lysate or tissue lysate;

(b) purifying endogenous JNK/SAPKs;

(c) carrying out a kinase reaction with the JNK/SAPKs obtained according to step (b) in the presence of ATP and GST transcription factor protein containing one or both N-terminal phosphorylation sites; and

(d) detecting the amount of phosphorylated TST transcription factor protein by adding a specific anti-p-GST transcription factor antibody to the mixture from step (c) and determining the activity of JNK/SAPKs by means of the amount of bound anti-p-GST transcription factor antibodies.

The term “transcription factor” shall refer to the below special substrates for JNK/SAPKs, e.g. (TF)Elk-1, c-Jun, Jun B, Jun D and ATF-2, in particular c-Jun.

A cell lysate for obtaining the JNK/SAPKs can be prepared by means of routine methods known to a person skilled in the art, e.g. by means of the method described in the below example. The person skilled in the art is also familiar with all of the measures which have to be taken so that the JNK/SAPKs are fully and actively available when the cells are opened. For example, the degradation by proteases can be suppressed by means of known protease inhibitors, such as PMSF.

In order to be able to determine the activity of JNK/SAPKS, it is advantageous for the endogenous JNK/SAPKS from the cell lysate to be enriched so as to separate the endogenous transcription factor proteins. This can be done by methods known to the person skilled in the art, e.g. by means of immune precipitation or classical protein purification methods.

In a preferred embodiment of the method according to the invention, the JNP/SAPKs are enriched and/or isolated from the cell lysate by means of immune precipitation. This is done according to standard methods using anti-JNK/SAPK antibodies bound e.g. to a column or beads, e.g. magnetic beads. Here, the antibodies suited for this method are monoclonal, polyclonal or synthetic antibodies. The term “antibody” used herein also comprises fragments. In this connection, the term “fragment” refers to all parts of the monoclonal antibody (e.g. Fab, Fv or single chain Fv fragments) which have an epitope specificity the same as that of the complete antibody. The person stilled in the art is familiar with the production of such fragments. The antibodies according to the invention are preferably monoclonal antibodies. The antibodies according to the invention can be produced according to standard methods, JNK/SAPK peptides or synthetic fragments thereof preferably serving as an immunogen. These peptides and the fragments thereof can be produced by obtaining the corresponding gene, cloning and recombinant expression. Methods of obtaining monoclonal antibodies are known to the person skilled in the art.

In a particularly preferred embodiment of the method according to the invention the endogenous JNK/SAPKs contained in the cell lysate are immunoprecipitated by incubating them with specific antibodies and then binding them to protein A-sepharose beads. In this connection, the antibodies should be directed against JNK/SAPKs or against an epitope which all JNK/SAPKs have in common so that the assay is quantitative, i.e. the overall activity of all JNK/SAPKs is detected. The further method steps can then be taken with the JNK/SAPKs released by the protein A-sepharose beads or with the still bound JNK/SAPKs, in any case, it has to be ensured that the biological activity of JNK/SAPKs is neither inhibited nor otherwise negatively affected by binding to the antibody.

In a further, even more preferred embodiment of the method according to the invention, the specific antibodies are anti-JNK1 anti-JNK2 and/or anti-JNK3 antibodies which are directed against common epitopes of JNK1, JNK2 and JNK3 (Davis et al., Biochem. Soc. Sympl. 1999, 64, pp. 1-12). The above described antibodies are commercially available.

The invention is described below by the example of the transcription factor c-Jun. However, this should not be understood to be a limitation of the invention. As to the other above listed transcription factors analogous steps should be taken.

The kinase reaction with the obtained JNK/SAPKs is carried out in the presence of ATP and GST-c-Jun containing one or both N-terminal phosphorylation sites. Conditions for kinase reactions are known to the person skilled in the art, and the reaction can be carried out e.g. according to the below example. The skilled person himself can also purify the GST-c-Jun required for this reaction step For prepare it recombinantly (van Dam et al., EMBO J. 1995, 14: 1798-1811). Suitable GST-c-Jun protein is also commercially available (see in this connection the statements made in the below example). GST-c-Jun is a c-Jun protein which is expressed recombinantly in bacteria and fused to GST. GST enables the purification of the recombinant c-Jun protein by means of glutathione sepharose beads.

The term “GST-c-Jun” comprises all forms of the protein still accessible to a phosphorylation reaction by JNK/SAPKs and thus also forms which are modified as compared to the natural forms, e.g. a different glycosylation pattern, substitution, deletion and/or addition of amino acid(s), etc. It also comprises shortened forms of GST-c-Jun, wherein the shortened forms may be shortened at the C-terminus or N-terminus, in any case, still have at least one phosphorylation site, preferably they still have both phosphorylation sites.

In the most preferred embodiment of the method according to the invention, the GST-c-Jun used for the kinase reaction is GST-c-Jun 1-166 or GST c-Jun-1-79, which can be bought from the Santa Cruz company, Heidelberg, Germany, the numbers referring to the positions of the remaining amino acids.

An antibody suited for the detection of the phosphorylated GST-c-Jun can be prepared according to common methods, the above statements on anti-JNK antibodies applying analogously as regards this antibody and/or the production thereof. This antibody must comply with two main preconditions: It must bind specifically to GST-c-Jun and be able to distinguish between the phosphorylated and non-phosphorylated forms, i.e. it only binds to the phosphorylated form. Such antibodies may be produced e.g. by using phosphorylated GST-c-Jun (preferably containing both phosphorylation sites) or a fragment containing the phosphorylated binding site(s) (e.g. Ser63 and/or Ser73) as an immunogen and then screening for antibodies which only bind to the phosphorylated form of GST-c-Jun but not to the non-phosphorylated one.

Regarding the method according to the invention the concentration range of GST-c-Jun is preferably between 0.2 and 5 μg per 50 μl kinase reaction, the range of 1-3 μg/50 μl kinase reaction being preferred. The concentration range of the cell lysate used is preferably between 50 and 1000 jig protein per 50 μl kinase reaction, the range of 100-300 μg being preferred. The anti-p-GST-c-Jun antibody is preferably available in excess as compared to GST-c-Jun to obtain a quantitative measurement, the concentration of the antibody depending on its affinity.

The (absolute) JNK/SAPK activity may be determined quantitatively by comparison with a control reaction containing JNK/SAPKs having known activity.

In a further preferred embodiment of the method according to the invention the anti-p-GST-c-Jun antibody recognizes specifically the phosphorylation to Ser73 and/or Ser63.

The activity of JNK/SAPKs can be determined by binding the anti-p-GST-c-Jun antibody using various general methods, e.g. Western blot, immunofluorescence, ELISA, radioimmunoassay (RIA), etc., Western blot being preferred for the method according to the invention. The anti-p-GST-c-Jun antibody preferably has a detectable labeling, with a fluorescent compound, a colloidal metal, a chemiluminescent compound, a bioluminescent compound, a phosphorescent compound or an enzyme being particularly preferred as the labeling. In the most preferred embodiment of the method according to the invention, the JNK/SAPK activity is determined by a primary polyclonal rabbit anti-p-GST-c-Jun antibody and a commercially available secondary anti-rabbit horseraddish peroxidase antibody and by increased chemiluminescence. The anti-p-GST-c-Jun antibody may also be absorbed to a solid carrier by means of common methods. In addition, there is the possibility that the primary antibody is biotin-conjugated and a streptavidin-peroxidase-linked secondary antibody is used. If a determination in the fluorescence microscope or FACS is desired, a fluorescein-linked or rhodamine-linked secondary antibody is preferably used.

The present invention also provides kits which are of use for the method according to the invention and preferably contain an anti-p-GST-c-Jun antibody or a fragment thereof and a GST-c-Jun protein containing one or both phosphorylation sites as well as an antibody by which JNK1, JNK2 and/or JNK3 can be enriched by immune precipitation. The kit can also contain JNK/SAPks or active parts thereof, e.g. for the purpose of control or accurate quantification, and protein A-sepharose beads for immune precipitation. Depending on the development of the kit, the antibody may be conjugated to another unit, e.g. a labeling and/or it may be immobilized on a solid carrier (substrate). The kit may also contain a second antibody for identifying p-GST-c-Jun/antibody complexes. The antibody or the fragment thereof may be freely available or be immobilized to a solid carrier, e.g. a plastic dish, a test tube, a microtitration plate, a test rod, etc. The kit may also contain instructions explaining the use of the included compounds in an assay for determining the JNK/SAPK activity. The kit may also contain suitable reagents for detecting labelings or for labeling positive and negative controls, wash solutions, ECL reagents (Amersham company, Braunschweig, Germany), dilution buffers, etc.

The method according to the invention is also of use for applications in which a cancer therapy based on the activation of JNK/SAPKs is carried out. Thus, the present invention also relates to a method of assessing the effectiveness of a cancer therapy, characterized by determining the therapeutic ability of activating JNK/SAPKs by the above described method according to the invention, preferably at various times in the course of the therapy.

Another advantage of the method according to the invention is that JNK/SAPKs are fully immunoprecipitated from a cell lysate. Therefore, it is not only possible to analyze by this method the phosphorylation of c-Jun but the phosphorylation of all substrates of JNK/SAPks selectively. These substrates comprise e.g. the transcription factors (TCF)/Elk-1, c-Jun, Jun B. Jun D and ATF-2. The phosphorylated proteins are detected by an antibody specifically detecting the phosphorylated form of the corresponding transcription factors.

### BRIEF DESCRIPTION OF THE FIGURES

FIG. 1: Diagram as to the assay according to the invention

JNK/SAPKs are immunoprecipitated with specific antibodies (FL, C-17, Santa Cruz company), bound to protein A-sepharose and subsequently incubated with non-radioactive ATP and GST-c-Jun protein. The activity of JNK/SAPKs results in the phosphorylation of the GST-c-Jun protein to Ser-63 and Ser-73. The amount of phosphorylation reflecting the activity of JNK/SAPKs can be detected by Western blot analysis using a phospho-c-Jun-specific antibody.

FIG. 2: Specific phosphorylation of GST-c-Jun 1-166 in the assay according to the invention

Lysates of JURKAT cells which had been treated with cisplatin (1 μg/ml) for 4 hours to activate JNK/SAPKs were used for the immune precipitation of JNK/SAPKs with the polyclonal rabbit-IgG antibodies FL and C17 (Santa Cruz company) and protein A-sepharose. The kinase reaction was carried out by incubation of the immunoprecipitate with kinase buffer and unlabeled ATP (lane a) or with kinase buffer, unlabeled ATP and GST-c-Jun 1-1661 as a substrate (lane d). Alternatively, the kinase buffer was only incubated with GST-c-Jun 1-166 and unlabeled ATP without immunoprecipitate (lane b). The various reaction mixtures or only kinase buffers (lane c) were separated by means of 12% SDS-PAGE and analyzed using Western blot. The molecular weight (in kDa) is shown on the left-hand side of the figure. The phosphorylation of the GST-c-Jun protein was detected by a polyclonal phospho-c-Jun-Ser73-specific IgG antibody (New England BioLabs) and by increased chemiluminescence. Explanation as to the method: Peroxidase-conjugated secondary antibody is bound to the primary antibody with increased chemilimunescence. If a solution containing the substrate “luminol” is added, luminol is decomposed by the peroxidase and a photoreaction results. The amount of light forming can be detected quantitatively on an X-ray film or in a luminometer.

The following example explains the invention.

### EXAMPLE 1

### Non-Radioactive Detection of JNK/SAPK Activity

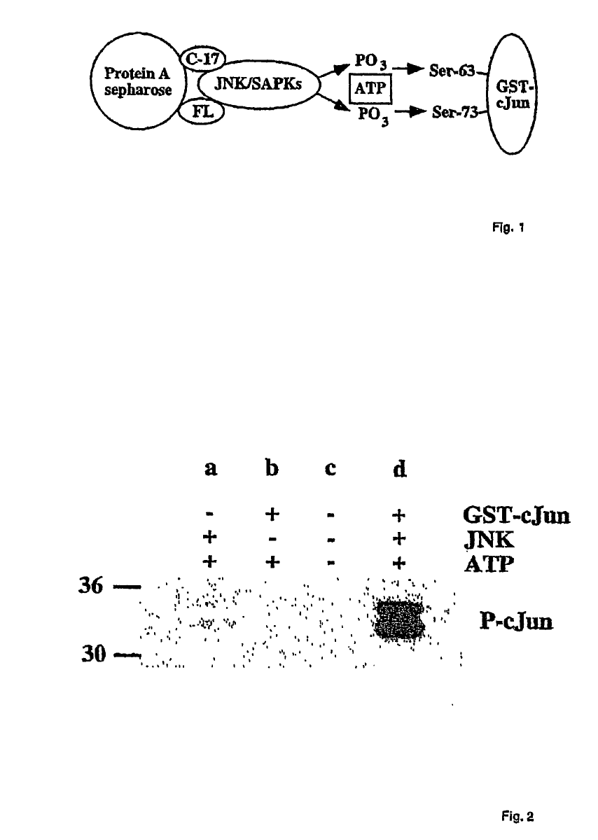

A total of 1×10⁷ JURKAT cells (cell library of Deutsches Krebsforschungszentrum, Heidelberg; Culturing at 37° C. in an incubator in RPMI-1640 medium containing 10% heat-inactivated fetal calf serum (Biochrom company, Hamburg, Germany), 25 mM HEPES and 2 mM L-glutamine (GIBCO/Life Technologies company, Eggenstein, Germany) were washed in icecold PBS and then lyzed in 400 μl flag buffer (20 mM Tris, pH 7.5, 137 mM NaCl, 2 mM EDTA, 1% Triton X-100™, 10% glycerol, 25 mM β-glycerolphosphate, 2 mM sodium pyrophosphate, 1 mm Na₃VO₄, pH 10.0, 1 mM PMSF, 10 μg/ml aprotinin, 10 μg/ml leupeptine). The lysates were clarified by centrifugation (20 min. 2° C., 13,000 rpm) and the nucleus-free supernatant which only contained the proteins of the cytoplasm and the membranes was transferred to a new vessel. After determined the protein content there was added in each case 200 μg protein with 1.5 μl polyclonal IgG rabbit antibody against JNK1 and JNK3 (C-17, sc-474; Santa Cruz company, Heidelberg, Germany; directed against the amino acids 369 to 384 of the C-terminus) or directed against JNK1, JNK2 and JNK3 (FL, Santa Cruz company, amino acids 1 to 384 of JNK1) and the mixture was mixed while rotated at room temperature for 45 min. Thereafter, 25 μl of a suspension of protein A-sepharose beads (Sigma, Deisenhofen, Germany) were added, and the mixture was incubated while rotated at room temperature for 1 hour. In order to remove non-specifically bound proteins, the beads were washed twice with flag buffer and once with kinase buffer. The kinase reaction was carried out in a total volume of 50 μl in the presence of non-labeled ATP (100 μm) and GST-c-Jun fusion protein (2 μg) containing the two N-terminal phosphorylation sites Ser63 and Ser73 which are essential for the phosphorylation by JNK/SAPKs (FIG. 1). For this purpose, the protein A-sepharose beads were suspended in 50 μl kinase buffer consisting of 25 mM HEPES, pH 7.4, 25 mM MgCl₂, 25 mM β-glycerophosphate, 100 μM ATP (Roche, Mannheim, Germany), 0.1 mM Na₃VO₄ and 2 μg GSt-c-Jun 1-166, which had been prepared as described by van Dam et al., EMBO J. 14 (1995), 1798-1811, or alternatively 2 μg TST-c-Jun 1-79 (Santa Cruz). Following incubation at 37° for 25 min, the reaction was concluded by adding 30 μl 3×SDS-application buffer (187.5 mM Tris-HCl, pH 6.8 6% SDS, 30% glycerol, 10% β-mercaptoethanol, 0.3% bromophenol blue). The products were separated by means of 12; SDS-PAGE under reducing conditions and applied to ECL membranes (Amersham, Braunschweig, Germany). Phosphorylated GST-c-Jun protein was stained using a polyclonal rabbit IgG-phospo-specific anti-c-Jun(Ser73) antibody (New England Biolabs) which was used at a dilution of 1:5,000. This antibody permitted the specific determination of the JNK/SAPK-induced phosphorylation of c-Jun to Ser73, this site being important for the c-Jun-dependent transcriptional activity. Bound antibodies were detected by anti-rabbit horseraddish peroxidase conjugate (Santa Cruz, Heidelberg) at a dilution of 1:5,000 and by means of increased chemiluminescence. The specificity of the assay was determined by carrying out the kinase reaction in the absence or presence of GST-c-Jun, immunoprecipitate or ATP. Only the kinase reaction which contained all reagents together yielded a strong signal (FIG. 2). A weak band visible in the reaction without GST-c-Jun (lane a) might have resulted from the phosphorylation of endogenous Jun protein which precipitated together with JNK/SAPKs. The double band corresponds to monophosphorylated and biphosphorylated Jun protein. This assay only required development periods within seconds and showed very little background binding. It is thus highly sensitive and it can be assumed that this assay is more sensitive by several orders than the former assays based on 32P labeling.

1. A method of determining the activity of JNK/SAPKs, wherein the method comprises the steps of:
(a) preparing a cell lysate or tissue lysate;
(b) purifying endogenous JNK/SAPKs;
(c) carrying out a kinase reaction with the JNK/SAPKs obtained according to step (b) in the presence of ATF and GST transcription factor protein containing one or both N-terminal phosphorylation sites;
(d) detecting the amount of phosphorylated GST transcription factor protein by adding a specific anti-p-GST transcription factor antibody to the mixture from step (c) and determining the activity of JNK/SAPKs by means of the amount of bound anti-p-GST transcription factor antibodies.

2. The method according to claim 1, wherein in step (b) the endogenous JNK/SAPKs contained in the cell or tissue lysate are isolated from the lysate by means of immune precipitation.

3. The method according to claim 2, wherein the endogenous JNP/SAPKs are isolated by incubating them with specific antibodies and then binding them to protein A-sepharose beads.

4. The method according to claim 3, wherein the specific antibodies are anti-JNK1, anti-JNK2 and/or anti-JNK3 antibodies.

5. The method according to any of claims 1 to 4, wherein the transcription factor is (TCF)/Elk-1, c-Jun, Jun B, Jun D or ATF-2.

6. The method according to any of claims 1 to 5, wherein GST-c-Jun containing both N-terminal phosphorylation sites is used in step (c).

7. The method according to claim 6, wherein the GST-c-Jun is GST-c-Jun 1-166 or GST c-Jun 1-79.

8. The method according to any of claims 1 to 7, wherein in step (d) the antibody identifies specifically the phosphorylation of c-jun to Ser73 and/or Ser63.

9. The method according to any of claims 1 to 8, wherein in step (d) the amount of bound antibody is determined by means of Western blot.

10. The method according to any of claims 1 to 9, wherein the antibody carries a detectable labeling.

11. The method according to claim 10, wherein the labeling is a fluorescent compound, a colloidal metal, a chemiluminescent compound, a bioluminescent compound, a phosphorescent compound or an enzyme.

12. The method of evaluating the effectiveness of a cancer therapy, characterized by measuring the therapeutic ability of activating the JNK/SAPKs in a sample according to a method as defined in any of claims 1 to 11.

13. A kit for carrying out the method according to any of claims 1 to 12, containing a GST-c-Jun protein containing one or both N-terminal phosphorylation sites and an anti-p-GST-c-Jun antibody.
